(19) United States
(12) Patent Application Publication
(10) Pub. No.: US 2008/0025926 A1
(43) Pub. Date: Jan. 31, 2008

(54) Oral care formulations with hydrogen peroxide and lycopene

(75) Inventors: Nicholas M. Kavouklis, Tampa, FL (US); Rosemary A. Gaudreault, Park Ridge, IL (US)

Correspondence Address: BUCHANAN, INGERSOLL & ROONEY PC, POST OFFICE BOX 1404, ALEXANDRIA, VA, 22313-1404, US

(73) Assignee: Dr. Nick's White & Healthy, LLC, Tampa, FL (US)

(21) Appl. No.: 11/491,187

(22) Filed: Jul. 24, 2006

(51) Int. Cl. A61K 8/49; A61K 8/22

(52) U.S. Cl. 424/53

(57) ABSTRACT

A toothpaste formulation is disclosed which comprises hydrogen peroxide; and cetylpyridinium chloride, and wherein the hydrogen peroxide and the cetylpyridinium chloride have been combined in the toothpaste formulation for not more than about seven minutes prior to administration to an individual. A mouth rinse formulation is disclosed which comprises hydrogen peroxide; and cetylpyridinium chloride, and wherein the hydrogen peroxide and the cetylpyridinium chloride have been combined in the mouth rinse formulation for not more than about seven minutes prior to administration to an individual. A whitening gel formulation is disclosed which comprises hydrogen peroxide; and pH adjuster, and wherein the hydrogen peroxide and the pH adjuster have been combined in the whitening gel formulation for not more than about seven minutes prior to administration to an individual. The toothpaste formulation, the mouth rinse formulation, and the whitening gel formulation further comprise lycopene.

### FIELD OF THE INVENTION

The present invention includes oral care formulations. The oral care formulations include toothpaste formulations, mouthwash or mouth rinse formulations, and whitening gel formulations.

### BACKGROUND OF THE INVENTION

Over the years, numerous consumer products have been manufactured and marketed for cleansing and whitening of teeth. The products have taken the form of toothpastes, powders, gels, and liquid rinses. Some have been compounded in solid or powder form for mixing with water, which is then used as a rinse.

Toothpastes and rinses often have an unsatisfactory taste and/or they include synthetic sweeteners. Further, toothpastes and rinses suffer from limited exposure time and contact between teeth and whitening agent, and exposure time is further diminished when taste is unsatisfactory.

More recently, solid composite structures have been employed to administer whitening compositions to the teeth. Those structures often employ a solid substrate to which is affixed a dental whitening and/or cleansing formulation. The dental whitening and/or cleansing formulation is in the form of a paste or gel that is reversibly applied to a major surface of the solid substrate as a thin layer. The solid substrate is a plastic sheet or other pliable material that can be readily deformed by the user to complement the shape and contours of the users' teeth. The surface of the substrate bearing the dental whitening and/or cleansing formulation is placed in contact with the users teeth, thereby imparting the whitening and/or cleansing effect. The use of such substrates is laborious, uncomfortable, and time consuming.

Cetylpyridinium chloride is a cationic quaternary ammonium compound that is known to have antibacterial properties and helps fight plaque and gingivitis (see 21 C.F.R. § 356). Cetylpyridinium chloride can be purchased in two forms: cetylpyridinium chloride, USP (Anhydrous) CAS Number 123-03-5 and cetylpyridinium chloride monohydrate, USP CAS Number 6004-24-6. The latter form contains approximately 5 wt % water.

Cetylpyridinium chloride monohydrate is incompatible with strong oxidizing or reducing agents and anionic detergents. This may be a reason why cetylpyridinium chloride is not used as an active ingredient in oral care formulations.

Most oral care formulations such as toothpaste formulations contain anionic surfactants to increase their wetting capability. Hydrogen peroxide is used as a whitening agent in oral care formulations. However, cetylpyridinium chloride will react with the anionic surfactants and the hydrogen peroxide rendering them ineffective. Therefore, there is a need for oral care formulations that can include both cetylpyridinium chloride and hydrogen peroxide thereby embodying the qualities of both ingredients in one oral care formulation without any substantial loss of efficacy upon use by an individual.

### SUMMARY OF THE INVENTION

According to a first embodiment, a toothpaste formulation is disclosed. The toothpaste formulation comprises hydrogen peroxide; and cetylpyridinium chloride, and wherein the hydrogen peroxide and the cetylpyridinium chloride have been combined in the toothpaste formulation for not more than about seven minutes prior to administration to an individual.

The hydrogen peroxide is present in an amount of about 2.5 wt % to about 7.5 wt %. In another embodiment, the hydrogen peroxide is present in an amount of about 4.4 wt %. The cetylpyridinium chloride is present in an amount of from about 0.1 wt % to about 0.2 wt %. In another embodiment, the cetylpyridinium chloride is present in an amount of from about 0.14 wt %.

The pH of the toothpaste formulation is in a range of from about 5.5 to about 7.5.

According to a second embodiment, a mouth rinse formulation is disclosed. The mouth rinse formulation comprises hydrogen peroxide; and cetylpyridinium chloride, and wherein the hydrogen peroxide and the cetylpyridinium chloride have been combined in the mouth rinse formulation for not more than about seven minutes prior to administration to an individual.

The hydrogen peroxide is present in an amount of from about 2.5 wt % to about 7.5 wt %. In another embodiment, the hydrogen peroxide is present in an amount of about 4.3 wt %. The cetylpyridinium chloride is present in an amount of from about 0.1 wt % to about 0.2 wt %. In another embodiment, the cetylpyridinium chloride is present in an amount of about 0.14 wt %.

The pH of the mouth rinse formulation is in a range of from about 5.5 to about 8.5.

The mouth rinse formulation can further comprise flavor selected from the group consisting of methyl salicylate, menthol, cinnamic aldehyde, clove oil, substantially pure natural mint oil, wintergreen flavor, peppermint flavor, spearmint flavor, and mixtures thereof.

According to a third embodiment, a whitening gel formulation is disclosed. The whitening gel formulation comprises hydrogen peroxide; and pH adjuster, and wherein the hydrogen peroxide and the pH adjuster have been combined in the whitening gel formulation for not more than about seven minutes prior to administration to an individual.

The whitening gel formulation can further comprise flavor enhancers selected from the group consisting of sodium citrate and potassium citrate.

The pH adjuster adjusts the pH of the whitening gel from about 6.5 to about 9.0. In one embodiment, the hydrogen peroxide is present in an amount of from about 3 wt % to about 15 wt %. In another embodiment, the hydrogen peroxide is present in an amount of about 8 wt %.

The pH adjusters have a pH in a range of from about 7.5 to about 9.5. The whitening gel formulation can be dispensed onto a mouth tray for administration to the individual.

According to a fourth embodiment, a container for dispensing a toothpaste formulation is disclosed. The container comprises a first chamber having hydrogen peroxide; and a second chamber having cetylpyridinium chloride, and wherein the first chamber and the second chamber are configured such that when a dispensing mechanism is activated the hydrogen peroxide and the cetylpyridinium chloride are combined to form the toothpaste formulation.

The hydrogen peroxide and the cetylpyridinium chloride have been combined in the toothpaste formulation for not more than about seven minutes prior to administration to an individual.

The hydrogen peroxide is present in an amount of from about 2.5 wt % to about 7.5 wt %. In another embodiment, the hydrogen peroxide is present in an amount of about 4.4 wt %. The cetylpyridinium chloride is present in an amount of about 0.1 wt % to about 0.2 wt %. In another embodiment, the cetylpyridinium chloride is present in an amount of about 0.14 wt %.

The pH of the toothpaste formulation is in a range of from about 5.5 to about 7.5. The first chamber can further comprise additives selected from the group consisting of water, phosphoric acid, block copolymer of polyethylene glycol and polypropylene glycol, glycerin, and mixtures thereof. The second chamber can further comprise additives selected from the group consisting of water, hydrated silica, glycerin, sorbitol, xylitol, mannitol, potassium citrate, sodium citrate, titanium dioxide, flavor, triticum vulgare (wheat) bran extract, lycopene, sodium hydroxide, cellulose gum, xanthan gum, and mixtures thereof.

According to a fifth embodiment, a container for dispensing a mouth rinse formulation is disclosed. The container comprises a first chamber having hydrogen peroxide; and a second chamber having cetylpyridinium chloride, and wherein the first chamber and the second chamber are configured such that when a dispensing mechanism is activated the hydrogen peroxide and the cetylpyridinium chloride are combined to form the mouth rinse formulation.

The hydrogen peroxide and the cetylpyridinium chloride have been combined in the mouth rinse formulation for not more than seven minutes prior to administration to an individual.

The hydrogen peroxide is present in an amount of from about 2.5 wt % to about 7.5 wt %. In another embodiment, the hydrogen peroxide is present in an amount of about 4.3 wt %. The cetylpyridinium chloride is present in an amount of from about 0.1 wt % to about 0.2 wt %. In another embodiment, the cetylpyridinium chloride is present in an amount of about 0.14 wt %.

The pH of the mouth rinse formulation is in a range of from about 5.5 to about 8.5. The first chamber can further comprise additives selected from the group consisting of water, phosphoric acid, and mixtures thereof. The second chamber can further comprise additives selected from the group consisting of water, glycerin, sorbitol, xylitol, mannitol, potassium citrate, sodium citrate, flavor, triticum vulgare (wheat) bran extract, lycopene, and mixtures thereof.

According to a sixth embodiment, a container for dispensing a whitening gel formulation is disclosed. The container comprises a first chamber having hydrogen peroxide; and a second chamber having pH adjusters, and wherein the first chamber and the second chamber are configured such that when a dispensing mechanism is activated the hydrogen peroxide and the pH adjusters are combined to form the whitening gel formulation.

The hydrogen peroxide and the pH adjusters have been combined in the whitening gel formulation for not more than about seven minutes prior to administration to an individual.

The second chamber can further comprise flavor enhancers selected from the group consisting of sodium citrate and potassium citrate. The pH adjusters adjust the pH of the whitening gel to from about 6.5 to about 9.0. The pH adjusters have a pH in a range of from about 7.5 to about 9.5.

The hydrogen peroxide is present in an amount of from about 3 wt % to about 15 wt %. In another embodiment, the hydrogen peroxide is present in an amount of about 8 wt %.

The whitening gel formulation is dispensed onto a mouth tray for administration to the individual. The first chamber can further comprise additives selected from the group consisting of water, phosphoric acid, block copolymer of polyethylene glycol and polypropylene glycol, glycerin, and mixtures thereof. The second chamber can further comprise additives selected from the group consisting of water, block copolymer of polyethylene glycol and polypropylene glycol, glycerin, sorbitol, xylitol, mannitol, potassium citrate, sodium citrate, polyethylene sorbitan monooleate, flavor, triticum vulgare (wheat) bran extract, lycopene, sodium hydroxide, and mixtures thereof.

According to a seventh embodiment, a method of administering an oral care formulation from a container having a first chamber and a second chamber is disclosed. The method comprises (a) activating a dispensing mechanism to dispense hydrogen peroxide from the first chamber and cetylpyridinium chloride from the second chamber to obtain the oral care formulation; and (b) administering the oral care formulation of (a) to an individual such that the hydrogen peroxide and the cetylpyridinium chloride have been combined for not more than about seven minutes prior to administration.

The oral care formulation can be a toothpaste formulation and/or a mouth rinse formulation.

The hydrogen peroxide is present in an amount of from about 2.5 wt % to about 7.5 wt %. The cetylpyridinium chloride is present an amount of about 0.1 wt % to about 0.2 wt %. In another embodiment, the cetylpyridinium chloride is present in an amount of about 0.14 wt %

In one embodiment, when the oral care formulation is a toothpaste formulation, the hydrogen peroxide is present in an amount of about 4.4 wt %. In another embodiment, when the oral care formulation is a mouth rinse formulation, the hydrogen peroxide is present in an amount of about 4.3 wt %.

The pH of the oral care formulation is in a range of from about 5.5 to about 8.5.

In one embodiment, when the oral care formulation is a toothpaste formulation, (i) the first chamber can further comprise additives selected from the group consisting of water, phosphoric acid, block copolymer of polyethylene glycol and polypropylene glycol, glycerin, and mixtures thereof, and/or (ii) the second chamber can further comprise additives selected from the group consisting of water, hydrated silica, glycerin, sorbitol, xylitol, mannitol, potassium citrate, sodium citrate, titanium dioxide, flavor, triticum vulgare (wheat) bran extract, lycopene, sodium hydroxide, cellulose gum, xanthan gum, and mixtures thereof.

In another embodiment, when the oral care formulation is a mouth rinse formulation, (i) the first chamber can further comprise additives selected from the group consisting of water, phosphoric acid, and mixtures thereof, and/or (ii) the second chamber can further comprise additives selected from the group consisting of water, glycerin, sorbitol, xylitol, mannitol, potassium citrate, sodium citrate, flavor, triticum vulgare (wheat) bran extract, lycopene, and mixtures thereof.

According to an eighth embodiment, a method of administering a whitening gel formulation from a container having a first chamber and a second chamber is disclosed. The method comprises (a) activating a dispensing mechanism to dispense hydrogen peroxide from the first chamber and pH adjusters from the second chamber to obtain the whitening gel formulation; and (b) administering the whitening gel formulation of (a) to an individual such that the hydrogen peroxide and the pH adjusters have been combined for not more than about seven minutes prior to administration.

The second chamber can further comprise flavor enhancers selected from the group consisting of sodium citrate and potassium citrate.

The pH adjusters adjust the pH of the whitening gel to from about 6.5 to about 9.0 and the pH adjusters have a pH in a range of from about 7.5 to about 9.5.

In one embodiment, the hydrogen peroxide is present in an amount of from about 3 wt % to about 15 wt %. In another embodiment, the hydrogen peroxide is present in an amount of about 8 wt %.

The whitening gel formulation is dispensed onto a mouth tray for administration to the individual. The first chamber can further comprise additives selected from the group consisting of water, phosphoric acid, block copolymer of polyethylene glycol and polypropylene glycol, glycerin, and mixtures thereof. The second chamber can further comprise additives selected from the group consisting of water block copolymer of polyethylene glycol and polypropylene glycol, glycerin, sorbitol, xylitol, mannitol, potassium citrate, sodium citrate, polyethylene sorbitan monooleate, flavor, triticum vulgare (wheat) bran extract, lycopene, sodium hydroxide, and mixtures thereof.

### DETAILED DESCRIPTION OF THE INVENTION

Definitions

“Teeth”, as used herein, is meant to include natural teeth, and any other hard surfaces, such as crowns, caps, fillings, bridges, dental implants, and the like, that are permanently fixed within the oral cavity and cleansed in situ within the oral cavity.

“Oral care formulation”, as used herein, is meant to include a single-phase oral care formulation or may be a combination of two or more oral compositions delivered in multiple phases. The oral composition is a product that, in the ordinary course of usage, is not intentionally swallowed for purposes of systemic administration of particular therapeutic agents, but rather, is retained in the oral cavity for a time sufficient to contact substantially all of the dental surfaces and/or oral tissues for purposes of oral activity.

“Container”, as used herein, is meant to include a jar, cup, can, tube, aerosol can, tub, pump, bottle or any other liquid holding or dispensing means having one or chambers that physically separate various formulation components from one another until the time of use.

“Safe and effective amount” or “effective amount”, as used herein, are meant an amount of an agent (e.g., anti-calculus agent) high enough to significantly improve the condition to be treated, but low enough to avoid serious side effects (at a reasonable benefit/risk ratio), within the scope of sound medical/dental judgment. The safe and effective amount of an agent (e.g., anti-calculus agent) may vary with the particular condition being treated, the age and physical condition of the patient being treated, the severity of the condition, the duration of treatment, the nature of concurrent therapy, the specific form of the source employed, and the particular vehicle from which the agent is applied.

“Tartar”, “calculus”, or “plaque”, as used herein, are used interchangeably and refer to mineralized dental plaque biofilms.

“Unit dose form”, as used herein, refers to physically discrete units suitable as unitary dosages for human subjects and other mammals, each containing a predetermined quantity of active material calculated to produce the desired therapeutic effect. Therefore, any method of administration described herein is meant to administer a unit dose of the oral care formulation.

“Individual”, as used herein, is meant to include a vertebrate. In various embodiments, vertebrates include mammals. Mammals include, but are not limited to, humans, rodents (i.e., mice, rats, and hamsters), farm animals, sport animals and pets. In another embodiment, the individual is a mammal. In still another embodiment, the individual is a human.

“Dispensing mechanism”, as used herein, is meant any actuating mechanism or valve or other mechanism that functions to dispense components of an oral care formulation located in at least two chambers. Examples of devices suitable for dispensing multi-component products such as those disclosed herein are described in U.S. Pat. Nos. 4,850,517; 5,051,305; 5,271,530; 5,337,929; 5,429,279; 5,443,569; 5,918,771; and 6,220,483, and references cited therein, all of which are incorporated herein by reference. Such devices can be used not only to discharge the contents from the container, but also to create or enhance foaming of the product. Devices useful in packaging and dispensing the products of the present invention are also commercially available from Airspray International B.V., Netherlands.

“Hydrogen peroxide”, as used herein, is meant to include any hydrogen peroxide that can be used in oral care formulations or by an individual for a primary purpose of whitening teeth. The hydrogen peroxide can be obtained from any commercial source such as Degussa Peroxide Ltd. In one embodiment, hydrogen peroxide can be substituted with other similar peroxides. It is to be understood that one of ordinary skill in the art can substitute equivalent amounts of other peroxides including but not limited to organic peroxides such as urea peroxide, glyceryl peroxide and benzoyl peroxide as well as metal ion containing peroxides such as calcium peroxide.

“Toothpaste” or “toothpaste formulation”, as used herein, are meant to include a paste-like or gel-like formulation used to clean and improve the aesthetic appearance and health of teeth.

“Mouthwash formulation”, “mouth rinse formulation”, “mouthwash”, or “mouth rinse”, as used herein, are all meant to include oral formulations used for oral hygiene by typically swishing or gargling for about half a minute and then spitting out. Therefore, the mouthwash or mouth rinse formulations are substantially liquid in form.

“Whitening gel” or “whitening gel formulation”, as used herein, are meant to include any gel-like or paste-like formulations which serve a primary purpose of whitening teeth due to the presence of oxidizing agents such as hydrogen peroxide.

A dental cleansing and whitening formulation is compounded using ingredients selected to increase the residence time of the formulation in and around the teeth and gums. The increased residence time of the active agents and whitener on the teeth and gums provides improved cleansing, disinfecting, and whitening. The formulation can be compounded in the form of a paste or a liquid rinse. One or the other of those compositions can be formulated and/or dispensed to produce a foam.

Further, the rinse and/or the paste can be prepared using only natural sweeteners and flavor agents.

The formulations of the present invention, whether paste or rinse, include an aqueous solvent, such as water, various active agents for plaque and decay prevention, and dimethyl isosorbide; the foregoing mixture can then be combined with a whitening agent, typically at the time of use.

Dimethyl isosorbide has not been used as here in oral care products, but proves effective in holding the active agents close to the teeth and gums to prolong and enhance the whitening effect.

The formulation can further comprise a Methyl Vinyl Ether/Maleic Anhydride Copolymer to provide better mouth feel and further contribute to increased residence time of active agents in contact with teeth and gums. Such agents also control the formation and growth of tartar, by preventing or reducing degradation of pyrophosphate in the mouth. Suitable co-polymers include those commercially available from International Specialty Products (Wayne, N.J.) within the Gantrez® family (e.g., Gantrez S®)

The formulation can further comprise one or more humectants. Suitable humectants are known in the oral care art, and selection of type and quantity are within the capabilities of one of ordinary skill in the art. Examples include sorbitol, NC sorbitol, glycerin, propylene glycol, mannitol, hydrolyzed corn starch, and combinations thereof.

Suitable active agents can be selected from the group consisting of sodium benzoate, xylitol, cetylpyridinium chloride, and combinations thereof. Other dental cleansing active agents that may be used, alternatively or in combination with any or all of the foregoing, include sodium fluoride, sodium monofluorophosphate, and potassium nitrate.

Various agents can also be incorporated for imparting flavor, or masking distasteful components. These agents can be all natural and sugar-free. Examples of agents used to modify, improve, or mask flavor of an oral care product such as this include polyethoxylated castor oil (e.g., PEG-40 hydrogenated castor oil such as Cremophor RH 40®), maltol, and combinations thereof.

The instant formulations can further include emulsifiers such as propylene glycol and/or various hydrogels. In one embodiment, the hydrogel is a block copolymer of polyethylene glycol and polypropylene glycol (e.g., Poloxamer 407).

Embodiments of the formulations of the invention can also include antioxidants, which are well known to those skilled in this art. Examples include tocopherol acetate and/or ascorbic acid.

Of the foregoing components, the humectant can be included to a concentration of about 5%-30% (by weight of Phases A&B, as described below). The xylitol can be included to a range of about 0.1%-2.0% by weight. Sodium benzoate can be introduced to about 0.01%-0.90% by weight. The Methyl Vinyl Ether/Maleic Anhydride Copolymer co-polymer can be about 0.01-0.10% by weight.

The formulations can comprise one or more of various derivatives of sorbitol anhydride. These can be incorporated to about 0.1% to about 2.0% by weight. Examples include polysorbate 20, polysorbate 80, sorbitan sesquioleate, dimethyl isosorbide, and polysorbate 60. Dimethyl isosorbide provides good penetration of actives cetylpyridinium chloride and sodium benzoate into oral mucosa, tooth, and tooth-gingiva junction.

Without wishing to be bound by any theory, it appears that the use of dimethyl isosorbide alone or in combination with xylitol affords unexpected improvement in the efficacy of cleansing, and decay and plaque prevention, as well as in enhanced whitening. Further, the combination of dimethyl isosorbide appears to provide unexpected improvement in cleansing and whitening when combined with sodium benzoate and cetylpyridinium chloride.

In addition, the formulations of the invention can include a flavor component. Many suitable flavor agents and mixtures are known in the art of oral care formulations. Such flavor components are commercially available from sources such as Mafco (Camden, N.J.), Noville (South Hackensack, N.J.) and others. One example is Noville AN 147298.

Other excipients and additives can be added such as diluents, solvents, preservatives, antioxidants, buffers, biocides, and the like.

The foregoing components can be combined at the time of use with a whitening agent. The whitening agent can be an oxidizing agent that can be selected from the group of hydrogen peroxide, carbamide peroxide, sodium chlorite, sodium hypochlorite, and chlorine dioxide. In addition, the formulation provides additional active agents, such as sodium benzoate, xylitol, and cetylpyridinium chloride. Dimethyl isosorbide can be added to the formulation. Dimethyl isosorbide has been found to impart an advantageous penetration and coating effect, particularly between teeth and at the tooth-gingiva junction, in these formulations.

In one series of embodiments, the formulation of the present invention comprises a whitening agent of a dilute solution of about 2% to about 10% peroxide. In another embodiment, the whitening agent is about 4% to about 8% peroxide. Yet another embodiment comprises a whitening agent of about 6% hydrogen peroxide.

In one embodiment, the invention provides a dual component dental cleansing and whitening composition comprising: a first component of an aqueous solvent, dimethyl isosorbide, and active agents for plaque and decay prevention; and a second component comprising a whitening agent. The whitening agent is also referred to as an oxidizing agent, and can be hydrogen peroxide, carbamide peroxide, sodium chlorite, sodium hypochlorite, chlorine dioxide, and/or combinations thereof. The two components are packaged and/or stored separately. The two components can be mixed upon being dispensed from the container, or at the time of use. By mixing or combining the two components at the time of use, a high level of activity of the oxidizing agent is maintained.

In another embodiment, the composition comprises: a first component of an aqueous solvent, dimethyl isosorbide, and an active agent selected from the group consisting of sodium benzoate, xylitol, cetylpyridinium chloride, sodium fluoride, sodium monofluorophosphate, potassium nitrate, and combinations thereof; and a second component comprising an oxidizing agent selected from the group consisting of hydrogen peroxide, carbamide peroxide, sodium chlorite, sodium hypochlorite, chlorine dioxide, and combinations thereof.

In yet another embodiment, the dual component composition comprises a first component comprising an aqueous solvent, dimethyl isosorbide, xylitol, sodium benzoate, cetylpyridinium chloride; and a second component comprising an oxidizing agent selected from the group consisting of hydrogen peroxide, carbamide peroxide, sodium chlorite, sodium hypochlorite, chlorine dioxide, and combinations thereof.

The present invention further includes a method for cleansing and whitening the teeth of a mammal comprising administering to the mammal's oral cavity, and particularly the teeth and gums, a composition comprising a mixture of a first component of an aqueous solvent, dimethyl isosorbide, and active agents for plaque and decay prevention; and a second component comprising a whitening agent.

The dual component composition of the instant invention can be compounded and/or packaged such that the composition foams. The composition itself, and particularly when produced as a foam, has greater residence time in and around the teeth and gums. This enhanced residence time imparts more effective cleansing and whitening effect than other compositions, e.g., those formulated as a rinse or a paste.

In one embodiment, the formulation of the present invention is dispensed through a dual chambered device. The dual chambered device is configured such that the components of the respective chambers are isolated from each other until the formulation is dispensed at time of use. The device dispenses the Actives/Flavoring component (combined Phases A&B) in about a 1:1 weight (or volume) ratio with the whitening component.

A suitable dispensing mechanism is any device that permits the controlled emission of two components so that they are substantially combined upon exiting their respective chambers of the dispensing mechanism. Thus, any two-chamber mechanism having a common port or adjacent ports for dispensing the two components can be used. An example of one such mechanism is a dual chambered syringe type mechanism as is commonly used for dispensing epoxy adhesives. Thus, any device having a common actuator for emitting material from two or more separate chambers in about equal volume or mass can be used for this purpose.

The common port or proximal ports should be configured to provide thorough mixing of the two components as they are emitted from the respective chambers.

In an alternative embodiment, other dispensing means can be employed as where the dispensing of the material from the respective chambers is either effected through the aid of gases, or wherein suitable gas is infused into the product as it is emitted from the respective or collective chambers. Examples of devices suitable for dispensing multi-component products such as those disclosed herein are described in U.S. Pat. Nos. 4,850,517; 5,051,305; 5,271,530; 5,337,929; 5,429,279; 5,443,569; 5,918,771; and 6,220,483, and references cited therein, all of which are incorporated herein by reference. Such devices can be used not only to discharge the contents from the container, but also to create or enhance foaming of the product. Devices useful in packaging and dispensing the products of the present invention are also commercially available from Airspray International B.V., Netherlands.

In one embodiment, the formulations of the present invention are compounded with the following ingredients: Purified Water, 50-55% (Solvent); Sodium Benzoate, 0.1-0.5% (Plaque Prevention); Xylitol, 0.1-1.0% (Plaque and Decay Prevention); Mannitol, 0.01-0.5% (Flavor Enhancer); Cetylpyridinium Chloride, 0.01-0.5% (Antiplaque and Antigingival Agent); Dimethyl Isosorbide, 0.1-1.0% (Helps Deliver Actives); Gantrez S-97, 0.01-0.1% (Helps Hold Actives To Gingiva); Cocomidopropyl Betaine (30%), 0.5-5.0% (Mild Surfactant); Sorbitol, 5.0-25.0% (Humectant); Glycerin, 5.0-25.0% (Humectant); hydrogenated castor oil (e.g., PEG-40 hydrogenated castor oil), 1.0-10.0% (Flavor Enhancer And Emulsifier); Propylene Glycol, 1.0-20.0% (Humectant & Emulsifier); Flavor (Noville, An147298), 0.5-5.0% (Flavor); Poloxamer 407, 0.05-2.5% (Emulsifier); Tocopherol Acetate, 0.01-1.0% (Antioxidant); and Ascorbic/PG, 0.01-1.0% (Antioxidant). Unless stated otherwise, all percentages expressed herein are percent by weight.

As discussed elsewhere, the foregoing constituents are first compounded as two separate phases (also referred to herein as Phases A & B), which are eventually combined into a single mixture. The resulting single mixture is packaged in one chamber of a multi-chamber package or dispensing mechanism.

Another chamber of the package or dispensing mechanism is charged with the whitening agent, e.g., hydrogen peroxide or carbamide peroxide. In one embodiment, the whitening agent is about a 6% solution of hydrogen peroxide. As discussed elsewhere herein, the dispensing mechanism effects a thorough mixing of the contents of the various chambers of the package. In one embodiment, the dispensing mechanism is a dual chamber package that mixes the contents of the two chambers in about a 1:1 ratio (by weight). Accordingly, the resulting formulation as used has about a 3% by weight composition of hydrogen peroxide.

Carbamide peroxide can also be used as the whitening agent. Carbamide peroxide is about 35% hydrogen peroxide, and so a mixture of about 10 to 30% carbamide peroxide would be suitable. Other whitening agents are known in the oral care art, and suitable quantities and concentrations can be determined without undue experimentation by one of ordinary skill in the art.

One advantage of the dual component formulation of the invention is that the separation of the components until time of use avoids decomposition or deactivation of the whitening agent. For example, it is known that exposure of hydrogen peroxide to organic materials and other agents leads to decomposition of the peroxide, and loss of activity. Thus, the present invention affords a dual component dental whitening formulation comprising one component that comprises a mixture of active agents and another component that comprises a dental whitening agent, and wherein said components are maintained in separate containers and are combined at the time of use.

The following include some exemplary formulations. In each case, the formulation of the table is compounded first in separate phases A and B. The two phases are combined in a single chamber of a dual chamber dispensing mechanism.

The procedure for preparing and combining the Actives sub-mix (Phase A) and the Flavor sub-mix (Phase B) was as follows:

### 1.) To the formula amount of water add the specified quantities of sodium benzoate and cetylpyridinium chloride;

### 2.) Mix until these two ingredients are completely dissolved;

### 3.) Add the thickener (e.g., Gantrez S-97) and, while continuing to mix, heat the blend to 45° C.;

### 4.) Hold and mix at 45° C. for 10 minutes to allow the thickener (e.g., Gantrez) to fully hydrate;

### 5.) Cool to room temperature;

### 6.) While cooling, add xylitol, maltol, dimethyl isosorbide, cocamodipropyl betaine, sorbitol and glycerin with mixing;

### 7.) Mix until the solution becomes homogeneous;

### 8.) Add the emulsifier (e.g., Poloxamer 407) to the main blend and mix until dissolved;

### 9.) In another vessel mix the components of Phase B (Flavor sub-mix): flavor, propylene glycol, Cremophor RH-40®, tocopherol acetate and ascorbic/PG;

### 10.) Continue mixing until achieving a homogeneous solution;

### 11.) Slowly add the Flavor sub-mix (Phase B) solution to the Actives sub-mix (Phase A);

### 12.) Continue mixing for 15 minutes after completing the addition of the flavor sub mix.

The resulting mixture is suitable for use, and can be packaged for commercial sale. In one embodiment, the combined mixture of Phases A&B is packaged in a sealed container. The sealed container can be configured or combined as part of a dual chamber dispensing mechanism. The dual chamber dispensing mechanism facilitates the simultaneous distribution of combined Phases A&B and a whitening agent or component.

The whitening agent, e.g., hydrogen peroxide and/or carbamide peroxide, is charged into another chamber of the dispensing mechanism. In each of the Exemplary Rinse Formulations 1-5, the whitening agent used was a 6% solution of hydrogen peroxide.

The dispensing mechanism used in the following examples mixes the two components of the respective chambers in about a 1:1 weight ratio. On mixing, the whitening agent is diluted by about 50%, such that a 3% solution of hydrogen peroxide is administered.

EXEMPLARY RINSE FORMULATION # 1

| | INGREDIENTS | WEIGHT % | USE |
|---|---|---|---|
| PHASE A | Water, Purified | 52.5000% | Solvent |
| | Sodium Benzoate | 0.2500% | Plaque Prevention |
| | Xylitol | 0.5000% | Plaque And Decay Prevention |
| | Maltol | 0.1000% | Flavor Enhancer |
| | Cetylpyridinium Chloride | 0.1000% | FDA Approved Antiplaque And |
| | | | Antigingival Agent |
| | Dimethyl Isosorbide | 0.5000% | Helps Deliver Actives |
| | Gantrez S-97 | 0.0500% | Helps Hold Actives To Gingiva |
| | Cocomidipropyl Betaine | 1.8000% | Mild Surfactant |
| | (30%) | | |
| | Sorbitol | 15.0000% | Humectant |
| | Glycerin | 15.0000% | Humectant |
| PHASE B | PEG-40 hydrogenated | 4.0000% | Flavor Enhancer And Emulsifier |
| | castor oil | | |
| | Propylene Glycol | 8.0000% | Humectant & Emulsifier |
| | Flavor (Noville, An147298) | 1.5000% | Flavor |
| | Poloxamer 407 | 0.5000% | Emulsifier |
| | Tocopherol Acetate | 0.1000% | Antioxidant |
| | Ascorbic/PG | 0.1000% | Antioxidant |
| | | 100.0000% | |

EXEMPLARY RINSE FORMULATION #2

| | INGREDIENT | WEIGHT % |
|---|---|---|
| PHASE A | Water, Purified | 54.3500% |
| | Sodium Benzoate | 0.5000% |
| | Xylitol | 15.0000% |
| | Maltol | 0.1000% |
| | Gantrez S-97 | 0.0500% |
| | Cocomidipropyl Betaine (30%) | 1.8000% |
| | Glycerin | 15.0000% |
| PHASE B | PEG-40 hydrogenated castor oil | 0.5000% |
| | Propylene Glycol | 10.0000% |
| | Flavor (Noville, An147298) | 1.5000% |
| | Poloxamer 407 | 1.0000% |
| | Tocopherol Acetate | 0.1000% |
| | Ascorbic/PG | 0.1000% |
| | | 100.0000% |

EXEMPLARY RINSE FORMULATION # 3

| | INGREDIENT | WT % |
|---|---|---|
| PHASE A | Water, Purified | 53.0000% |
| | Sodium Benzoate | 0.2500% |
| | Xylitol | 0.5000% |
| | Maltol | 0.1000% |
| | Cetylpyridinium Chloride | 0.1000% |
| | Gantrez S-97 | 0.0500% |
| | Cocomidipropyl Betaine | 1.8000% |
| | (30%) | |
| | Sorbitol | 15.0000% |
| | Glycerin | 15.0000% |
| PHASE B | PEG-40 hydrogenated | 4.0000% |
| | castor oil | |
| | Propylene Glycol | 8.0000% |
| | Flavor (Noville, | 1.5000% |
| | An147298) | |
| | Poloxamer 407 | 0.5000% |
| | Tocopherol Acetate | 0.1000% |
| | Ascorbic/PG | 0.1000% |
| | | 100.0000% |

EXEMPLARY RINSE FORMULATION # 4

| | INGREDIENT | WT % |
|---|---|---|
| PHASE A | Water, Purified | 50.8000% |
| | Sodium Benzoate | 0.2500% |
| | Xylitol | 11.1000% |
| | Maltol | 0.1000% |
| | Gantrez S-97 | 0.0500% |
| | Cetylpyridinium | 0.1000% |
| | Chloride | |
| | Dimethyl Isosorbide | 4.0000% |
| | Cocomidipropyl | 1.8000% |
| | Betaine (30%) | |
| | Glycerin | 14.0000% |
| PHASE B | PEG-40 hydrogenated | 4.0000% |
| | castor oil | |
| | Propylene Glycol | 8.1000% |
| | Polysorbate 20 | 3.0000% |
| | Flavor (Noville, | 1.5000% |
| | An147298) | |
| | Poloxamer 407 | 1.0000% |
| | Tocopherol Acetate | 0.1000% |
| | Ascorbic/PG | 0.1000% |
| | | 100.0000% |

EXEMPLARY RINSE FORMULATION # 5

| | INGREDIENT | WT % |
|---|---|---|
| PHASE A | Water, Purified | 50.8000% |
| | Sodium Benzoate | 0.2500% |
| | Xylitol | 11.1000% |
| | Maltol | 0.1000% |
| | Gantrez S-97 | 0.0500% |
| | Cetylpyridinium Chloride | 0.1000% |
| | Dimethyl Isosorbide | 4.0000% |
| | Cocomidipropyl Betaine | 1.8000% |
| | (30%) | |
| | Glycerin | 14.0000% |
| PHASE B | PEG-40 hydrogenated | 4.0000% |
| | castor oil | |
| | Propylene Glycol | 8.1000% |
| | Polysorbate 20 | 3.0000% |
| | Flavor (Noville, | 1.5000% |
| | An147298) | |
| | Poloxamer 407 | 1.0000% |
| | Tocopherol Acetate | 0.1000% |
| | Ascorbic/PG | 0.1000% |
| | | 100.0000% |

Oral care formulations are disclosed in the present invention. These oral care formulations can include cetylpyridinium chloride. Cetylpyridinium chloride is known to have antibacterial properties and helps fight plaque and gingivitis.

In one embodiment, the cetylpyridinium chloride can be cetylpyridinium chloride monohydrate. Cetylpyridinium chloride can be obtained from Spectrum Chemicals, Gardena, Calif.

Hydrogen peroxide is used in the oral care formulations of the present invention as a whitening agent as it is a strong oxidizing agent. However, cetylpyridinium chloride monohydrate is substantially incompatible with strong oxidizing or reducing agents and anionic detergents. In fact, if combined, reactions between cetylpyridinium chloride and anionic surfactants or hydrogen peroxide will render each component substantially ineffective for its purpose.

The oral care formulations of the present invention include toothpaste formulations, mouthwash or mouth rinse formulations, and whitening gel formulations.

The embodiments of the present invention utilize various ingredients in various amounts to form the oral care formulations described herein. A summary of the various ingredients is provided in Table 1. Table 1 also provides a short description of the primary purpose of the ingredient along with the primary source of the ingredient.

It should be noted, however, that the primary purpose and source of the ingredients described herein in the various embodiments are not meant to be limiting in any manner. It is to be understood that one of ordinary skill in the art will recognize that many if not all the ingredients described herein serve multiple purposes and can be derived from multiple sources either alone or in any combination.

TABLE 1
Ingredients in Oral Care Formulations

| Ingredient | Purpose | Source |
|---|---|---|
| Cetylpyridinium Chloride | Helps prevent and | Cationic detergent |
| | reduce plaque and | |
| | gingivitis | |
| Water | Consistency, diluent | Purified Water |
| Glycerin | Body, pleasant feel in the mouth | Vegetable based |
| Block copolymer of | Gelling Agent | Derivative of natural gas |
| polyethylene glycol and | | and oil |
| polypropylene glycol | | |
| (e.g., Poloxamer 407) | | |
| Hydrated Silica | Mild abrasive | Sand |
| Sorbitol | Sweetener | Edible fruit, corn |
| Xylitol | Sweetener | Plants, fruits, vegetables |
| Mannitol | Sweetener | Edible fruit, corn |
| Hydrogen Peroxide | Whitening Agent | Water with extra oxygen |
| | | molecule |
| Potassium Citrate | Controls pH, reduces | Potassium salt of Citric |
| | sensitivity of teeth in oral | Acid |
| | care products | |
| Sodium Citrate | Reduces astringency, | Sodium salt of Citric Acid |
| | enhances flavor | |
| Titanium Dioxide | Opacifier | Extracted from sands |
| Mint Oil | Flavor | Natural Mint Oil |
| Cellulose Gum | Thickening Agent | Polysaccharides |
| Xanthan Gum | Thickening Agent | Polysaccharides |
| Lycopene | Natural free radical | Derived from Tomatoes |
| | scavenger | |
| Triticum Vulgare (Wheat) | Anti-inflammatory effect | Derived from Wheat Bran |
| Bran Extract | | |
| Sodium Hydroxide | pH adjuster | Extracted from seawater |
| Phosphoric Acid | pH adjuster | Derived from |
| | | phosphorylated sugars |

Water acts as diluent in the oral care formulations providing the desired consistency.

Glycerin acts as a humectant in the oral care formulations. Glycerin can also adjust the viscosity of oral care formulations to the desired level. Moreover, glycerin leaves a pleasant feel in the mouth. When used in substantially high amounts, glycerin can also help to preserve the oral care formulations. Glycerin has a mild sweet flavor, which can be perceived as adding to consistency and flavor. The mouthwash or mouth rinse and the toothpaste formulations contain substantially high percentages of glycerin. Glycerin helps to maintain clarity of the mouth rinse formulation along with maintaining solubility of flavor(s) in the mouthwash or mouth rinse formulation. Glycerin helps maintain solubility of flavor(s) in a toothpaste formulation.

A block copolymer of polyethylene glycol and polypropylene glycol such as Poloxamer 407, which is a commercially available product, acts as a surfactant, solubilizer, and/or gelling agent in the oral care formulations. The block copolymer of polyethylene glycol and polypropylene glycol is derived from natural gas and oil.

Hydrated Silica is a mild abrasive, which gives oral care formulations such as toothpaste a smoother “gel” quality. A portion of the hydrated silica increases the viscosity of the toothpaste, and with the xanthan and cellulose gums forms a “gel” like product. A portion of the hydrated silica acts as a mild abrasive. Hydrated silica is a derivative of silica (i.e., silicon dioxide). Hydrated silica is an odorless, tasteless, white gelatinous substance, which is chemically inert.

Sorbitol can act as a humectant and/or a sweetener in the oral care formulations. Sorbitol works well with other ingredients and can have a synergistic effect on other sweeteners. Sorbitol is a naturally occurring ingredient found in many edible fruits and berries, corn, and seaweed.

Xylitol acts as a sweetener in the oral care formulations. Xylitol is a naturally occurring substance that can be found in plants, fruits, and vegetables and is produced in the human body by normal metabolism. Xylitol is the sweetest of the polyols and is just as sweet as sugar, but is not believed to promote tooth decay. Xylitol is believed to not raise insulin levels as much as sugar.

Mannitol acts as a sweetener and is a sugar alcohol in the oral care formulations. Mannitol has half the calories of sugar and is about half as sweet. The human body does not absorb Mannitol very easily, so it does not increase insulin levels as much as sugar. Mannitol is believed to not promote tooth decay. Mannitol and sorbitol are isomers of each other.

Without wishing to be bound by theory, it is believed that sorbitol, xylitol, and mannitol are sweeter than the sum of the individual sweeteners and results in synergistic blends, which provides a more pleasant taste to the oral care formulations described in the various embodiments.

Hydrogen Peroxide acts as a whitening agent in the oral care formulations because it is an effective oxidizing agent.

Potassium citrate modifies or enhances flavor, controls pH, and acts as a buffering agent in the oral care formulations. Potassium citrate chelates metal ions, adds body to artificially sweetened products, reduces sensitivity of teeth in oral care products, and is a synergist with antioxidants

Sodium citrate buffers pH, maintains stability of active ingredients, reduces astringency, acts as an antiplaque agent, chelates trace metal ions, and modifies or enhances flavor in the oral care formulations.

Titanium dioxide acts as an opacifier in the oral care formulations, especially in the toothpaste formulations. Titanium dioxide acts as a white pigment and scatters visible light, thereby imparting whiteness and brightness to oral care formulations such as toothpastes.

Flavor(s) can be added to the oral care formulations of the present invention. The flavor(s) can be selected from the group consisting of methyl salicylate, menthol, cinnamic aldehyde, clove oil, substantially pure natural mint oil, wintergreen flavor, peppermint flavor, spearmint flavor, and various combinations thereof. In one embodiment, the flavor is substantially pure natural mint oil, which imparts a mint flavor to the oral care formulations to which it is added.

Cellulose gum acts as a thickening agent, binder, stabilizer, protective colloid, suspending agent, rheology modifier, and/or flow control agent in the oral care formulations. Cellulose gum is substantially physiologically inert in the oral care formulations.

Xanthan gum acts as a thickening agent in the oral care formulations as it has a substantially high molecular weight polysaccharide produced by microbial fermentation.

Lycopene acts a natural free radical scavenger in the oral care formulations. Lycopene is derived from tomatoes and is a phytochemical (i.e., carotenoid) that inhibits mutagenesis and reduces nuclear damage. Lycopene also shows significant bioactivity as an antioxidant.

Triticum vulgare (wheat) bran extract in the oral care formulations supports healthy skin growth by high amounts of polyunsaturated fatty acids, tocopherols and beta-carotene. Triticum vulgare (wheat) bran extract restores skin tissue, supports wound healing, and has an anti-inflammatory effect.

Sodium hydroxide in the oral care formulations acts as a pH adjuster and can be extracted from seawater or other brines.

Phosphoric acid acts as a pH adjuster in the oral care formulations.

Polyethylene sorbitan monooleate (e.g., Polysorbate 80, which is commercially available) acts as an emulsifying agent.

### Toothpaste Formulation

In one embodiment, the oral care formulation is a toothpaste formulation.

In another embodiment, the toothpaste formulation is provided in a container having two chambers. In still another embodiment, the toothpaste formulation is provided in a container that is a dual chamber syringe.

A first chamber of the toothpaste container includes hydrogen peroxide. A second chamber of the toothpaste container includes cetylpyridinium chloride.

The dual chamber container configuration functions such that both the first chamber and the second chamber dispense their contents substantially simultaneously to be used substantially immediately so that the resulting mixture is contains an effective amount of hydrogen peroxide and an effective amount of cetylpyridinium chloride.

In one embodiment, the toothpaste formulation is used by an individual within about fifteen minutes of combining the hydrogen peroxide and the cetylpyridinium chloride. In another embodiment, the toothpaste formulation is used by an individual within about ten minutes of combining the hydrogen peroxide and the cetylpyridinium chloride. In still another embodiment, the toothpaste formulation is used by an individual within about seven minutes of combining the hydrogen peroxide and the cetylpyridinium chloride.

The effective amount of hydrogen peroxide in the first chamber is from about 1% to about 10% by weight and the cetylpyridinium chloride is present in the second chamber in an effective amount of about 0.01% to about 1% by weight. In another embodiment, the effective amount of hydrogen peroxide in the first chamber is from about 2.5% to about 7.5% by weight and the cetylpyridinium chloride is present in the second chamber in an effective amount of about 0.1% to about 0.2% by weight. In yet another embodiment, the hydrogen peroxide is present in the first chamber in an effective amount of about 4.4% by weight and the cetylpyridinium chloride is present in the second chamber in an effective amount of about 0.14% by weight.

In one embodiment, the pH of the dispensed toothpaste is about 5.5-7.5. In another embodiment, the pH of the dispensed toothpaste is about 6.5.

As discussed above, if the hydrogen peroxide is combined with cetylpyridinium chloride before use, the hydrogen peroxide will oxidize the cetylpyridinium chloride, and both compounds will have substantially diminished efficacy.

In one embodiment, the toothpaste formulation further comprises lycopene in an amount of up to about 1% by weight. In another embodiment, the toothpaste formulation further comprises lycopene in an amount of from about 0.01% to about 0.5% by weight. In still another embodiment, the toothpaste formulation further comprises lycopene in an amount of from about 0.01% to about 0.1% by weight.

In one embodiment, the toothpaste formulation has a formulation as set forth in Table 2.

TABLE 2
Toothpaste Formulation

| Chamber 1 | | Chamber 2 | |
|---|---|---|---|
| | % (by weight) | | % (by |
| | in | | weight) in |
| Component | Formulation | Component | Formulation |
| Hydrogen Peroxide | 1–10 | Water | 5–75 |
| Water | 20–70 | Cetylpyridinium | 0.01–1 |
| | | Chloride, USP | |
| Phosphoric Acid | 0–1 | Hydrated silica | 1–15 |
| | | 6.00% Zeodent 113 | |
| | | 4.25% Zeothix 265 | |
| Poloxamer 407 | 10–40 | Glycerin | 15–45 |
| Glycerin | 10–40 | Sorbitol | 1–10 |
| | | Xylitol | 1–10 |
| | | Mannitol | 1–10 |
| | | Potassium Citrate | 0.01–5 |
| | | Sodium Citrate | 0.01–5 |
| | | Titanium Dioxide | 0.01–5 |
| | | Flavor | 0.01–5 |
| | | Triticum vulgare | 0–1 |
| | | (wheat) bran extract | |
| | | Lycopene | 0–1 |
| | | Sodium Hydroxide | 0–1 |
| | | Cellulose Gum | 0.01–2 |
| | | Xanthan Gum | 0.01–2 |

### Whitening Gel

In one embodiment, the oral care formulation is a whitening gel.

In another embodiment, the whitening gel is provided in a container having a dual chamber. In still another embodiment, the whitening gel is provided in a dual chamber container that is a dual-chambered syringe.

In one embodiment, a first chamber of the container includes hydrogen peroxide in an effective amount of from about 3% to about 15% by weight. In another embodiment the effective amount of hydrogen peroxide in the first chamber is from about 6% to about 10% by weight. In still another embodiment, the effective amount of hydrogen peroxide in the first chamber is about 8% by weight.

In one embodiment, a second chamber of the container includes a pH adjuster having a pH in the range of about 7.5-9.5. In another embodiment, a second chamber of the container includes a pH adjuster having a pH in the range of about 8-9. In still another embodiment, a second chamber of the container includes a pH adjuster having a pH of about 8.3.

The dual chamber container configuration functions such that both the first chamber and the second chamber dispense their contents substantially simultaneously to be used substantially immediately so that the resulting mixture contains an effective amount of the pH adjuster and an effective amount of hydrogen peroxide.

In one embodiment, the whitening gel formulation is used by an individual within about fifteen minutes of combining the hydrogen peroxide and the pH adjuster. In another embodiment, the whitening gel formulation is used by an individual within about ten minutes of combining the hydrogen peroxide and the pH adjuster. In still another embodiment, the whitening gel formulation is used by an individual within about seven minutes of combining the hydrogen peroxide and the pH adjuster.

In one embodiment, the whitening gel is dispensed onto a mouth tray and used substantially immediately, so that the whitening gel includes an effective amount of about 1%-8% by weight of hydrogen peroxide and a pH of about 6.5 to about 9. In another embodiment, the whitening gel is dispensed onto a mouth tray and used substantially immediately, so that the whitening gel includes an effective amount of about 2%-6% by weight of hydrogen peroxide and a pH of about 7 to about 8. In still another embodiment, the whitening gel is dispensed onto a mouth tray and used substantially immediately, so that the whitening gel includes an effective amount of about 4.0% hydrogen peroxide and a pH of about 7.5.

If the hydrogen peroxide is combined with the pH adjuster before use, the pH of the hydrogen peroxide will increase, thus activating the hydrogen peroxide causing it to substantially lose its efficacy.

As hydrogen peroxide has a pH in the range of about 3.5-4.5, in order to activate hydrogen peroxide any chemical or combination of chemicals may be used to increase the pH of the hydrogen peroxide when combined. Ammonium, potassium and sodium hydroxides are materials generally used in the cosmetic industries to increase the pH up to about >11. This pH range is too harsh for the oral cavity and the mucous membranes. In contrast, if the pH is too low, i.e., too acidic, hydrogen peroxide can become too harsh for the oral cavity and the mucous membranes.

The pH adjuster used in the whitening gel can include any compound or a combination of compounds having a pH that is sufficiently high to counteract the low pH of the hydrogen peroxide. In one embodiment, the pH adjuster includes potassium citrate and/or sodium hydroxide.

In one embodiment, the whitening gel can be dispensed onto a mouth tray to be used by an individual.

In one embodiment, the whitening gel formulation further comprises lycopene in an amount of up to about 1% by weight. In another embodiment, the whitening gel formulation further comprises lycopene in an amount of from about 0.01% to about 0.5% by weight. In still another embodiment, the whitening gel formulation further comprises lycopene in an amount of from about 0.01% to about 0.1% by weight.

In one embodiment, the whitening gel formulation has a formulation as set forth in Table 3.

TABLE 3
Whitening Gel Formulation

| Chamber 1 | | Chamber 2 | |
|---|---|---|---|
| | % | | % (by |
| | (by weight) in | | weight) in |
| Component | Formulation | Component | Formulation |
| Hydrogen Peroxide | 3–15 | Water | 5–80 |
| Water | 25–70 | Poloxamer 407 | 10–40 |
| Phosphoric Acid | 0–1 | Glycerin | 1–15 |
| Poloxamer 407 | 10–40 | Sorbitol | 0.1–15 |
| Glycerin | 10–40 | Xylitol | 0.1–15 |
| | | Mannitol | 0.1–15 |
| | | Potassium Citrate | 0.1–10 |
| | | Sodium Citrate | 0.1–10 |
| | | Polysorbate 80 | 0–1 |
| | | Flavor | 0.01–5 |
| | | Triticum vulgare | 0–1 |
| | | (wheat) bran extract | |
| | | Lycopene | 0–1 |
| | | Sodium Hydroxide | 0–1 |

### Mouthwash or Mouth Rinse

In one embodiment, the oral care formulation is a mouthwash or mouth rinse formulation.

In one embodiment, the mouthwash or mouth rinse formulation is provided in a container having two chambers. In another embodiment, the mouthwash or mouth rinse formulation is provided in a container that is a bottle. A first chamber of the container includes a solution having hydrogen peroxide and a second chamber of the container includes a solution having cetylpyridinium chloride.

The dual chamber container configuration functions such that both the first chamber and the second chamber dispense their contents substantially simultaneously to be used substantially immediately so that the resulting mixture contains an effective amount of hydrogen peroxide and an effective amount of cetylpyridinium chloride.

In one embodiment, the mouthwash or mouth rinse formulation is used by an individual within about fifteen minutes of combining the hydrogen peroxide and the cetylpyridinium chloride. In another embodiment, the mouthwash or mouth rinse formulation is used by an individual within about ten minutes of combining the hydrogen peroxide and the cetylpyridinium chloride. In still another embodiment, the mouthwash or mouth rinse formulation is used by an individual within about seven minutes of combining the hydrogen peroxide and the cetylpyridinium chloride.

The effective amount of hydrogen peroxide in the first chamber is from about 1% to about 10% by weight and the cetylpyridinium chloride is present in the second chamber in an effective amount of about 0.01% to about 1% by weight. In another embodiment, the effective amount of hydrogen peroxide in the first chamber is from about 2.5% to about 7.5% by weight and the cetylpyridinium chloride is present in the second chamber in an effective amount of about 0.1% to about 0.2% by weight. In yet another embodiment, the hydrogen peroxide is present in the first chamber in an effective amount of about 4.3% by weight and the cetylpyridinium chloride is present in the second chamber in an effective amount of about 0.14% by weight.

In one embodiment, the pH of the dispensed mouth rinse is about 5.5-8.5. In another embodiment, the pH of the dispensed mouth rinse is about 6.5.

In one embodiment, the mouthwash or mouth rinse formulation further comprises lycopene in an amount of up to about 1% by weight. In another embodiment, the mouthwash or mouth rinse formulation further comprises lycopene in an amount of from about 0.01% to about 0.5% by weight. In still another embodiment, the mouthwash or mouth rinse formulation further comprises lycopene in an amount of from about 0.01% to about 0.1% by weight.

In one embodiment, the mouthwash or mouth rinse formulation has a formulation as set forth in Table 4.

TABLE 4
Mouthwash or Mouth Rinse Formulation

| Chamber 1 | | Chamber 2 | |
|---|---|---|---|
| | % | | % (by |
| | (by weight) in | | weight) in |
| Component | Formulation | Component | Formulation |
| Hydrogen Peroxide | 1–10 | Water | 10–90 |
| Water | 90–99 | Cetylpyridinium | 0.01–1 |
| | | Chloride, USP | |
| Phosphoric Acid | 0–1 | Glycerin | 1–45 |
| | | Sorbitol | 1–25 |
| | | Xylitol | 1–25 |
| | | Mannitol | 1–25 |
| | | Potassium Citrate | 0.01–5 |
| | | Sodium Citrate | 0.01–5 |
| | | Flavor | 0.01–5 |
| | | Triticum vulgare | 0–1 |
| | | (wheat) bran extract | |
| | | Lycopene | 0–1 |

### EXAMPLES

### Example 1—Toothpaste Formulation

### A. Preparation of Hydrogen Peroxide Gel for Toothpaste Formulation—

1. Thoroughly wash all equipment with hot, soapy water. Rinse with water until thoroughly clean.
2. Weigh and add #1 Distilled Water into vessel equipped with turbine mixer, sidesweeps and chiller. Begin chilling #1 to <5° C. (41° F.). The colder the solution is before adding #4 Pluracare® F 127 NF, the more readily #4 will go into solution.

### 3. Weigh and add #2 Glycerin, USP 99.7% FGK, mix until homogenous.

### 4. Weigh and add #3 Peralkali Hydrogen Peroxide, 35%. Mix until homogenous.

### 5. Increase turbine speed to form vortex and begin sidesweep agitation.

6. When the solution of #1-3 is at <5° C. (41° F.), add #4 Pluracare® F 127 NF slowly into vortex. Do not dump #4, as it will form clumps that are difficult to break. Once all of #4 is added, reduce speed of turbine (no vortex). Try not to incorporate any air. Mix for 2 hours. Check batch for any undissolved #4. Keep chilled at <5° C. (41° F.) until batch is emptied from vessel.
7. Continue mixing until #4 is completely dissolved. When #4 is completely dissolved, pull top and bottom samples, warm them to 25° C. (77° F.). Sample will become a gel. Keep remainder chilled. Determine pH.
8. Using remaining chilled sample, adjust pH to 3.80-4.20 with #5 Phosphoric Acid NF/FCC (10% Solution). Mix until homogenous. Warm sample to 25° C. (77° F.), and determine pH.
9. Weigh and add 75% of the amount #5 Phosphoric Acid NF/FCC (10% Solution) that was calculated in step 9 to the production batch. Mix until homogenous.
10. Take pH, and add additional Phosphoric Acid NF/FCC (10% Solution) to the batch as necessary. Mix until homogenous.
11. Pull top and bottom samples. Determine % Hydrogen Peroxide. (4.20-4.60%). Batch must be released in the middle of the specification.

### 12. Test according to other specifications.

### 13. Keep vessel covered at all times.

14. Fill according to specifications. If batch is aerated after completing, allow to remain in tank without mixing, other than sidesweep, and to maintain temperature of <5° C. (41° F.) or less.

### B. Preparation of Toothpaste Formulation—

1. Weigh and add #1 Distilled Water into vessel equipped with turbine, sidesweeps, heating and cooling ability. Warm #1 to 30°-32° C. (86°-90° F.).

### 2. Weigh and add #2 Sorbitol, mix until homogenous.

### 3. Weigh and add #3 Glycerin, USP 99.7% FGK, mix until homogenous.

4. Weigh and add #4 Maltisweet 3145, mix until homogenous. Maintain temperature at 30°-32° C. (86°-90° F.).

### 5. Increase turbine speed, but do not aerate while adding #5-7.

6. In a separate, clean, dry container, weigh and dry mix #5 CMC 7H4F, and #6 Keldent. Weigh and add #7 Glycerin, USP 99.7% FGK to dry mixed #5 and #6. Mix until homogenous. Immediately add to #14. Back wash any #5-7 remaining in mixing container. Product will become viscous and glossy. Mix for 30-40 minutes until fully hydrated. Batch will appear smooth, clear, and glossy, with a heavy viscosity when fully hydrated. If additional time is required to attain the correct appearance, continue mixing.

### 7. Increase temperature of batch to 36°-38° C. (97°-100°). If temperature is higher than 36°-38° C. (97°-100°), cool batch to 36°-38° C. (97°-100°).

8. Weigh and slowly add #8 Cornmint Oil Indian Terpeneless 40137-1. Mix until homogenous. Batch will be clear and viscous.

### 9. Weigh and slowly disperse #9 Zeodent 113. Mix 25-30 minutes or until homogenous.

10. Weigh and disperse #10 Xylitol-Crystalline. Mix 25-30 minutes or until homogenous.
11. Weigh and disperse #11 Tripotassium Citrate. Mix 15-20 minutes or until homogenous.

### 12. Weigh and disperse #12 Sodium Citrate F-6000. Mix 15-20 minutes or until homogenous.

13. Weigh and disperse #13 Titanium Dioxide AFDC 300. Mix 25-30 minutes or until homogenous. Batch will turn white and be viscous.

### 14. Cool #1-13 to 28°-29° C.

15. Weigh and add #14 Jarcopene 2% SG (5% Solution in Glycerin, USP 99.7% FGK). Mix until homogenous. Batch will become a light pink color.

### 16. Weigh and add #15 Wheat Bran Extract CO2 (5% Solution in Glycerin, USP 99.7% FGK). Mix until homogenous.

### 17. In a separate, clean container, weigh and add #16 Distilled Water warmed to 25°-26° C. (77°-79° F.). Weigh and add #17 Cetylpyridinium Chloride Monohydrate, USP. Mix until #17 is dissolved.

### 18. Add the solution of #16 and #17 to #1-15. Mix 30 minutes or until homogenous.

19. Weigh and disperse #18 Zeothix 265. Increase turbine and sidesweeps speed, if necessary, once all #18 is incorporated. Continue mixing 45-60 minutes, or until batch is smooth, viscous, and glossy. Do not aerate batch.
20. When batch is homogenous, pull top and bottom samples for pH determination in lab, and determine pH. Adjust pH to 7.00-7.60. Cool a sufficient sample to 25° C. (77° F.) to take pH. Sample will be viscous. Adjust pH to 7.00-7.60 with #19 Sodium Hydroxide NF/FCC (25% Solution). Mix until homogenous.
21. Weigh and add 75% of the calculated amount #19 Sodium Hydroxide NF/FCC (25% Solution) to the production batch. Mix until homogenous.
22. Take pH, and add additional Sodium Hydroxide NF/FCC (25% Solution) to the batch as necessary. Mix until homogenous.
23. Test batch according to specifications and keep vessel covered at all times. Fill according to specifications. Batch will gain in viscosity upon standing. It is best filled within 24 hours after completion.

TABLE 5
Toothpaste Formulation

| Chamber 1 | | Chamber 2 | |
|---|---|---|---|
| | % Present in | | % Present |
| Component | Formulation | Component | in Formulation |
| Hydrogen Peroxide | 4.4000 | Water | 32.0050 |
| Water | 54.5787 | Cetylpyridinium | 0.1400 |
| | | Chloride, USP | |
| Phosphoric Acid | 0.0213 | Hydrated silica | 10.2500 |
| | | 6.00% Zeodent 113 | |
| | | 4.25% Zeothix 265 | |
| Poloxamer 407 | 21.0000 | Glycerin | 37.0000 |
| Glycerin | 20.0000 | Sorbitol | 7.0000 |
| | | Xylitol | 4.5000 |
| | | Mannitol | 4.5000 |
| | | Potassium Citrate | 1.0000 |
| | | Sodium Citrate | 1.0000 |
| | | Titanium Dioxide | 1.0000 |
| | | Flavor | 0.8600 |
| | | Triticum vulgare | 0.0050 |
| | | (wheat) bran extract | |
| | | Lycopene | 0.0050 |
| | | Sodium Hydroxide | 0.0350 |
| | | Cellulose Gum | 0.5000 |
| | | Xanthan Gum | 0.2000 |

### Example 2—Whitening Gel Formulation

### A. Preparation of pH Adjuster for 8% Professional Strength Hydrogen Peroxide Gel for Whitening Gel Formulation—

1. Weigh and add #1 Distilled Water into vessel equipped with turbine mixer, sidesweeps and chiller. Begin chilling #1 to <5° C. (41° F.). The colder the solution is before adding #3 Pluracare® F 127 NF, the more readily #3 will go into solution.

### 2. Weigh and add #2 Glycerin, USP 99.7% FGK, mix until homogenous.

### 3. Increase turbine speed to form vortex and begin sidesweep agitation.

4. When the solution of #1 and #2 is at <5° C. (41° F.), weigh and add #3 Pluracare® F 127 NF slowly into vortex. Do not dump #3, as it will form clumps that are difficult to break. Once all of #3 is added, reduce speed of turbine (no vortex). Try not to incorporate any air. Mix 2 hours. Check batch for any undissolved #3. Keep chilled at <5° C. (41° F.) until batch is emptied from vessel.
5. Continue mixing until #3 is completely dissolved. Do not continue adding any raw materials until #3 is dissolved.
6. When #3 is completely dissolved, weigh and add #4 Sorbitol. Mix until homogenous.

### 7. Weigh and add #5 Maltisweet 3145. Mix until homogenous.

8. In a clean mixing vessel, weigh and add #6 Distilled Water. Weigh and add #7 Xylitol-Crystalline to #6. Mix until #7 is dissolved.
9. Add the solution from #6 and #7 to #1-5. Mix until homogenous. Batch will appear slightly syrupy in consistency. Do not proceed until batch is homogeneous.
10. Weigh and add #8 Jarcopene 2% SG (10% Solution in Tween 80K). Mix until homogenous. Batch will become a medium pink color.

### 11. Weigh and add #9 Wheat Bran Extract CO2 (10% Solution in Tween 80K). Mix until homogenous

### 12. Weigh and add #10 Cornmint Oil Indian Terpeneless 40137-1. Mix until homogenous.

13. Weigh and disperse #11 Tripotassium Citrate. Mix until dissolved. Batch will become slightly more viscous.
14. Weigh and disperse #12 Sodium Citrate F-6000. Mix until dissolved. Batch will become slightly more viscous.

### 15. Mix for an additional 30 minutes to insure that the batch is homogenous.

16. When batch is homogenous, pull top and bottom samples for pH determination in lab, and determine pH. Adjust pH to 8.00-8.60. Warm a sufficient sample to 25° C. (77° F.) to take pH. Sample will become a gel. Keep remainder chilled. Using chilled sample, adjust pH to 8.00-8.60 with #13 Sodium Hydroxide NF/FCC (25% Solution). Mix until homogenous. Warm sample to 25° C. (77° F.), and take pH.
17. Weigh and add 75% of the calculated amount #13 Sodium Hydroxide NF/FCC (25% Solution) to the production batch. Mix until homogenous.
18. Take pH, and add additional Sodium Hydroxide NF/FCC (25% Solution) to the batch as necessary. Mix until homogenous.

### 19. Test batch according to specifications.

### 20. Keep vessel covered at all times.

21. Fill according to specifications. If batch is aerated after completing, allow to remain in tank without mixing, other than sidesweep, and to maintain temperature of <5° C. (41° F.).

### B. Preparation of 8% Professional Strength Hydrogen Peroxide Gel in the Whitening Gel Formulation—

1. Thoroughly wash all equipment with hot, soapy water. Rinse with water until thoroughly clean.
2. Weigh and add #1 Distilled Water into vessel equipped with turbine mixer, sidesweeps and chiller. Begin chilling #1 to <5° C. (41° F.). The colder the solution is before adding #4 Pluracare® F 127 NF, the more readily #4 will go into solution.

### 3. Weigh and add #2 Glycerin, USP 99.7% FGK, mix until homogenous.

### 4. Weigh and add #3 Peralkali Hydrogen Peroxide, 35%. Mix until homogenous.

### 5. Increase turbine speed to form vortex and begin sidesweep agitation.

6. When the solution of #1-3 is at <5° C. (41° F.), add #4 Pluracare® F 127 NF slowly into vortex. Do not dump #4, as it will form clumps that are difficult to break. Once all of #4 is added, reduce speed of turbine (no vortex). Try not to incorporate any air. Mix 2 hours. Check batch for any undissolved #4. Keep chilled at <5° C. (41° F.) until batch is emptied from vessel.
7. Continue mixing until #4 is completely dissolved. When #4 is completely dissolved, pull top and bottom samples, warm them to 25° C. (77° F.). Sample will become a gel. Keep remainder chilled. Determine pH in lab.
8. Using remaining chilled sample, adjust pH to 3.80-4.20 with #5 Phosphoric Acid NF/FCC (10% Solution). Mix until homogenous. Warm sample to 25° C. (77° F.), and take pH.
9. Weigh and add 75% of the amount #5 Phosphoric Acid NF/FCC (10% Solution) that was calculated in step 9 to the production batch. Mix until homogenous.
10. Take pH, and add additional Phosphoric Acid NF/FCC (10% Solution) to the batch as necessary. Mix until homogenous.
11. Pull top and bottom samples. Determine % Hydrogen Peroxide (7.60-8.40%). Batch must be released in the middle of the specification.

### 12. Test according to other specifications.

### 13. Keep vessel covered at all times.

14. Fill according to specifications. If batch is aerated after completing, allow to remain in tank without mixing, other than sidesweep, and to maintain temperature of <5° C. (41° F.) or less.

TABLE 6
Whitening Gel Formulation

| Chamber 1 | | Chamber 2 | |
|---|---|---|---|
| | % Present in | | % Present |
| Component | Formulation | Component | in Formulation |
| Hydrogen Peroxide | 8.0000 | Water | 62.4896 |
| Water | 47.47875 | Poloxamer 407 | 20.5000 |
| Phosphoric Acid | 0.02125 | Glycerin | 9.000 |
| Poloxamer 407 | 23.0000 | Sorbitol | 2.1000 |
| Glycerin | 21.5000 | Xylitol | 1.7500 |
| | | Mannitol | 1.7500 |
| | | Potassium Citrate | 1.0000 |
| | | Sodium Citrate | 1.0000 |
| | | Polysorbate 80 | 0.0180 |
| | | Flavor | 0.4660 |
| | | Triticum vulgare | 0.0100 |
| | | (wheat) bran extract | |
| | | Lycopene | 0.0004 |
| | | Sodium Hydroxide | 0.0160 |

### Example 3—Mouthwash or Mouth Rinse Formulation

### A. Preparation of 4.3% Hydrogen Peroxide Solution for Mouth Rinse Formulation—

1. Thoroughly wash all equipment with hot, soapy water. Rinse with water until thoroughly clean.

### 2. Weigh and add #1 Distilled Water into vessel equipped with mixer.

### 3. Weigh and add #2 Peralkali Hydrogen Peroxide, 35%, mix until homogenous.

4. Pull top and bottom samples, and determine pH in lab. Adjust pH of the lab sample to 4.204.80 with #3 Phosphoric Acid NF/FCC (10% Solution) to determine amount required in the production batch of #3.
5. Weigh and add 75% of the calculated amount #3 Phosphoric Acid NF/FCC (10% Solution) to the batch. Mix until homogenous.
6. Take pH, and add additional Phosphoric Acid NF/FCC (10% Solution) to the batch as necessary. Mix until homogenous.

### 7. Determine % Hydrogen Peroxide. (4.10-4.50%). Batch must be released in the middle of the specification.

### 8. Test batch for remaining specifications.

### 9. Keep vessel covered at all times.

### 10. Fill according to specifications.

### B. Preparation of Mouth Rinse Formulation—

1. Weigh and add #1 Distilled Water into vessel equipped with turbine. Warm #1 to 32°-35° C. (90°-95° F.).

### 2. Weigh and disperse #2 Xylitol-Crystalline, mix until dissolved.

### 3. Weigh and add #3 Maltisweet 3145, mix until homogenous.

### 4. Weigh and add #4 Glycerin, USP 99.7% FGK, mix until homogenous.

5. Weigh and add #5 Sorbitol, mix until homogenous. Maintain temperature at 32°-35° C. (90°-95° F.).
6. In a separate DRY clean container equipped with mixer, weigh and add #6 Glycerin, USP 99.7% FGK. Heat to 42°-45° C. (108°-113° F.). Weigh and add #7 Cornmint Oil Indian Terpeneless 40137-1. Mix on high speed for 10-12 minutes or until homogenous. Immediately add the solution of #6 and #7 to #1-5. Mix #1-7 until homogenous and crystal clear.
7. Weigh and add #8 Jarcopene 2% SG (5% Solution in Glycerin, USP 99.7% FGK) warmed to 42°-45° C. (108°-113° F.). Mix until homogenous. Batch will become a light pink color.

### 8. Weigh and add #9 Wheat Bran Extract CO2 (5% Solution in Glycerin, USP 99.7% FGK) warmed to 42-45° C. (108°-113° F.). Mix until homogenous.

### 9. Weigh and add #10 Cetylpyridinium Chloride Monohydrate, USP. Mix until #10 is dissolved.

10. Weigh and disperse #11 Tripotassium Citrate. Mix 15-20 minutes or until homogenous.

### 11. Weigh and disperse #12 Sodium Citrate F-6000. Mix 15-20 minutes or until homogenous.

12. Keep vessel covered at all times. When batch is homogenous, pull top and bottom samples. Test batch according to specifications.

### 13. Fill according to specifications.

TABLE 7
Mouth rinse Formulation

| Chamber 1 | | Chamber 2 | |
|---|---|---|---|
| | % Active in | | % Active |
| Component | Formulation | Component | in Formulation |
| Hydrogen Peroxide | 4.30 | Water | 47.4775 |
| Water | 95.699 | Cetylpyridinium | 0.1400 |
| | | Chloride, USP | |
| Phosphoric Acid | 0.00100 | Glycerin | 29.0000 |
| | | Sorbitol | 13.300 |
| | | Xylitol | 4.0000 |
| | | Mannitol | 4.0000 |
| | | Potassium Citrate | 1.0000 |
| | | Sodium Citrate | 1.0000 |
| | | Flavor | 0.0750 |
| | | Triticum vulgare | 0.0050 |
| | | (wheat) bran extract | |
| | | Lycopene | 0.00250 |

While this invention has been particularly shown and described with references to various embodiments thereof, it will be understood by those skilled in the art that various changes in form and details may be made therein without departing from the spirit and scope of the invention as defined by the appended claims.

All references cited and/or discussed above are herein incorporated by reference in their entirety.

What is claimed is:

1. A toothpaste formulation comprising:
hydrogen peroxide; and
cetylpyridinium chloride, and
wherein the hydrogen peroxide and the cetylpyridinium chloride have been combined in the toothpaste formulation for not more than about seven minutes prior to administration to an individual.

2. The toothpaste formulation of claim 1, wherein (i) the hydrogen peroxide is present in an amount of about 2.5 wt % to about 7.5 wt %, (ii) the hydrogen peroxide is present in an amount of about 4.4 wt %, (iii) the cetylpyridinium chloride is present in an amount of from about 0.1 wt % to about 0.2 wt %, (iv) the cetylpyridinium chloride is present in an amount of about 0.14 wt %, and/or (v) the pH of the toothpaste formulation is in a range of from about 5.5 to about 7.5.

3. A mouth rinse formulation comprising:
hydrogen peroxide; and
cetylpyridinium chloride, and
wherein the hydrogen peroxide and the cetylpyridinium chloride have been combined in the mouth rinse formulation for not more than about seven minutes prior to administration to an individual.

4. The mouth rinse formulation of claim 3, wherein (i) the hydrogen peroxide is present in an amount of from about 2.5 wt % to about 7.5 wt %, (ii) the hydrogen peroxide is present in an amount of about 4.3 wt %, (iii) the cetylpyridinium chloride is present in an amount of from about 0.1 wt % to about 0.2 wt %, (iv) the cetylpyridinium chloride is present in an amount of about 0.14 wt %, and/or (v) the pH of the mouth rinse formulation is in a range of from about 5.5 to about 8.5.

5. The mouth rinse formulation of claim 3 further comprising flavor selected from the group consisting of methyl salicylate, menthol, cinnamic aldehyde, clove oil, substantially pure natural mint oil, wintergreen flavor, peppermint flavor, spearmint flavor, and mixtures thereof.

6. A whitening gel formulation comprising:
hydrogen peroxide; and
pH adjuster, and
wherein the hydrogen peroxide and the pH adjuster have been combined in the whitening gel formulation for not more than about seven minutes prior to administration to an individual.

7. The whitening gel formulation of claim 6, further comprising flavor enhancers selected from the group consisting of sodium citrate and potassium citrate.

8. The whitening gel formulation of claim 6, wherein (i) the pH adjuster adjusts the pH of the whitening gel from about 6.5 to about 9.0, (ii) the hydrogen peroxide is present in an amount of from about 3 wt % to about 15 wt %, (iii) the hydrogen peroxide is present in an amount of about 8 wt %, (iv) the pH adjusters have a pH in a range of from about 7.5 to about 9.5, and/or (v) the whitening gel formulation is dispensed onto a mouth tray for administration to the individual.

9. A container for dispensing a toothpaste formulation comprising:
a first chamber having hydrogen peroxide; and
a second chamber having cetylpyridinium chloride, and
wherein the first chamber and the second chamber are configured such that when a dispensing mechanism is activated the hydrogen peroxide and the cetylpyridinium chloride are combined to form the toothpaste formulation.

10. The container of claim 9, wherein (i) the hydrogen peroxide and the cetylpyridinium chloride have been combined in the toothpaste formulation for not more than about seven minutes prior to administration to an individual, (ii) the hydrogen peroxide is present in an amount of from about 2.5 wt % to about 7.5 wt %, (iii) the hydrogen peroxide is present in an amount of about 4.4 wt %, (iv) the cetylpyridinium chloride is present in an amount of about 0.1 wt % to about 0.2 wt %, (v) the cetylpyridinium chloride is present in an amount of about 0.14 wt %, (vi) the pH of the toothpaste formulation is in a range of from about 5.5 to about 7.5, (vii) the first chamber further comprises additives selected from the group consisting of water, phosphoric acid, block copolymer of polyethylene glycol and polypropylene glycol, glycerin, and mixtures thereof, and/or (viii) the second chamber further comprises additives selected from the group consisting of water, hydrated silica, glycerin, sorbitol, xylitol, mannitol, potassium citrate, sodium citrate, titanium dioxide, flavor, triticum vulgare (wheat) bran extract, lycopene, sodium hydroxide, cellulose gum, xanthan gum, and mixtures thereof.

11. A container for dispensing a mouth rinse formulation comprising:
a first chamber having hydrogen peroxide; and
a second chamber having cetylpyridinium chloride, and
wherein the first chamber and the second chamber are configured such that when a dispensing mechanism is activated the hydrogen peroxide and the cetylpyridinium chloride are combined to form the mouth rinse formulation.

12. The container of claim 11, wherein (i) the hydrogen peroxide and the cetylpyridinium chloride have been combined in the mouth rinse formulation for not more than seven minutes prior to administration to an individual, (ii) the hydrogen peroxide is present in an amount of from about 2.5 wt % to about 7.5 wt %, (iii) the hydrogen peroxide is present in an amount of about 4.3 wt %, (iv) the cetylpyridinium chloride is present in an amount of from about 0.1 wt % to about 0.2 wt %, (v) the cetylpyridinium chloride is present in an amount of about 0.14 wt %, (vi) the pH of the mouth rinse formulation is in a range of from about 5.5 to about 8.5, (vii) the first chamber further comprises additives selected from the group consisting of water, phosphoric acid, and mixtures thereof, and/or (viii) the second chamber further comprises additives selected from the group consisting of water, glycerin, sorbitol, xylitol, mannitol, potassium citrate, sodium citrate, flavor, triticum vulgare (wheat) bran extract, lycopene, and mixtures thereof.

13. A container for dispensing a whitening gel formulation comprising:
a first chamber having hydrogen peroxide; and
a second chamber having pH adjusters, and
wherein the first chamber and the second chamber are configured such that when a dispensing mechanism is activated the hydrogen peroxide and the pH adjusters are combined to form the whitening gel formulation.

14. The container of claim 13, wherein (i) the hydrogen peroxide and the pH adjusters have been combined in the whitening gel formulation for not more than about seven minutes prior to administration to an individual, (ii) the second chamber further comprises flavor enhancers selected from the group consisting of sodium citrate and potassium citrate, (iii) the pH adjusters adjust the pH of the whitening gel to from about 6.5 to about 9.0, (iv) the hydrogen peroxide is present in an amount of from about 3 wt % to about 15 wt %, (v) the hydrogen peroxide is present in an amount of about 8 wt %, (vi) the pH adjusters have a pH in a range of from about 7.5 to about 9.5, (vii) the whitening gel formulation is dispensed onto a mouth tray for administration to the individual, (viii) the first chamber further comprises additives selected from the group consisting of water, phosphoric acid, block copolymer of polyethylene glycol and polypropylene glycol, glycerin, and mixtures thereof, and/or (ix) the second chamber further comprises additives selected from the group consisting of water, block copolymer of polyethylene glycol and polypropylene glycol, glycerin, sorbitol, xylitol, mannitol, potassium citrate, sodium citrate, polyethylene sorbitan monooleate, flavor, triticum vulgare (wheat) bran extract, lycopene, sodium hydroxide, and mixtures thereof.

15. A method of administering an oral care formulation from a container having a first chamber and a second chamber, comprising:
(a) activating a dispensing mechanism to dispense hydrogen peroxide from the first chamber and cetylpyridinium chloride from the second chamber to obtain the oral care formulation; and
(b) administering the oral care formulation of (a) to an individual such that the hydrogen peroxide and the cetylpyridinium chloride have been combined for not more than about seven minutes prior to administration.

16. The method of administering the oral care formulation of claim 15, wherein the oral care formulation is a toothpaste formulation.

17. The method of administering the oral care formulation of claim 15, wherein the oral care formulation is a mouth rinse formulation.

18. The method of administering the oral care formulation of claim 15, wherein (i) the hydrogen peroxide is present in an amount of from about 2.5 wt % to about 7.5 wt %, (ii) the cetylpyridinium chloride is present in an amount of about 0.1 wt % to about 0.2 wt %, and/or (iii) the pH of the oral care formulation is in a range of from about 5.5 to about 8.5.

19. The method of administering the toothpaste formulation of claim 16, wherein (i) the first chamber further comprises additives selected from the group consisting of water, phosphoric acid, block copolymer of polyethylene glycol and polypropylene glycol, glycerin, and mixtures thereof, and/or (ii) the second chamber further comprises additives selected from the group consisting of water, hydrated silica, glycerin, sorbitol, xylitol, mannitol, potassium citrate, sodium citrate, titanium dioxide, flavor, triticum vulgare (wheat) bran extract, lycopene, sodium hydroxide, cellulose gum, xanthan gum, and mixtures thereof.

20. The method of administering the mouth rinse formulation of claim 17, wherein (i) the first chamber further comprises additives selected from the group consisting of water, phosphoric acid, and mixtures thereof, and/or (ii) the second chamber further comprises additives selected from the group consisting of water, glycerin, sorbitol, xylitol, mannitol, potassium citrate, sodium citrate, flavor, triticum vulgare (wheat) bran extract, lycopene, and mixtures thereof.

21. A method of administering a whitening gel formulation from a container having a first chamber and a second chamber, comprising:
(a) activating a dispensing mechanism to dispense hydrogen peroxide from the first chamber and pH adjusters from the second chamber to obtain the whitening gel formulation; and
(b) administering the whitening gel formulation of (a) to an individual such that the hydrogen peroxide and the pH adjusters have been combined for not more than about seven minutes prior to administration.

22. The method of administering the whitening gel formulation of claim 21, wherein (i) the second chamber further comprises flavor enhancers selected from the group consisting of sodium citrate and potassium citrate, (ii) the pH adjusters adjust the pH of the whitening gel to from about 6.5 to about 9.0, (iii) the hydrogen peroxide is present in an amount of from about 3 wt % to about 15 wt %, (iv) the hydrogen peroxide is present in an amount of about 8 wt %, (v) the pH adjusters have a pH in a range of from about 7.5 to about 9.5, (vi) the whitening gel formulation is dispensed onto a mouth tray for administration to the individual, (vii) the first chamber further comprises additives selected from the group consisting of water, phosphoric acid, block copolymer of polyethylene glycol and polypropylene glycol, glycerin, and mixtures thereof, and/or (viii) the second chamber further comprises additives selected from the group consisting of water block copolymer of polyethylene glycol and polypropylene glycol, glycerin, sorbitol, xylitol, mannitol, potassium citrate, sodium citrate, polyethylene sorbitan monooleate, flavor, triticum vulgare (wheat) bran extract, lycopene, sodium hydroxide, and mixtures thereof.
